SEQUENCE LISTING (16 sequences)

SEQ ID NO: 1 - protein, 18 aa, synthetic construct
MVKGKHHHHHHNGKGKSK

SEQ ID NO: 2 - protein, 88 aa, synthetic construct
MVKGKHHHHHHNGKGKSKGPRTLCGAELVDALQFVCGDRGFYFNKPTGYGSSSRRAPQTGIVDECCFRSCDLRRLEMYCAPLKPAKSA

SEQ ID NO: 3 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 4 - protein, 71 aa, synthetic construct
FVNQHLCGSHLVEALYLVCGDRGFYFNKPTGYGSSSRRAPQTGIVDECCFRSCDLRRLEMYCAPLKPAKSA

SEQ ID NO: 5 - protein, 83 aa, synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 6 - protein, 70 aa, Synthetic construct
GPRTLCGAELVDALQFVCGDRGFYFNKPTGYGSSSRRAPQTGIVDECCFRSCDLRRLEMYCAPLKPAKSA

SEQ ID NO: 7 - protein, 67 aa, synthetic construct
TLCGAELVDALQFVCGDRGFYFNKPTGYGSSSRRAPQTGIVDECCFRSCDLRRLEMYCAPLKPAKSA

SEQ ID NO: 8 - protein, 83 aa, synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 9 - protein, 83 aa, synthetic construct
MFPAMPLSSLFVNGPGTLCGAELVDALQFVCGDRGFYFNKPTGYGSSSRRAPQTGIVDECCFRSCDLRRLEMYCAPLKPAKSA

SEQ ID NO: 10 - protein, 21 aa, Homo sapiens
GIVEQCCTSICSLYQLENYCN

SEQ ID NO: 11 - protein, 30 aa, Homo sapiens
FVNQHLCGSHLVEALYLVCGERGFFYTPKT

SEQ ID NO: 12 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 13 - protein, 462 aa, synthetic construct
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFHAFVNQHLCGSHLVEALYLVCGDRGFYFNKPTGYGSSSRRAPQTGIVDECCFRSCDLRRLEMYCAPLKPAKSA

SEQ ID NO: 14 - protein, 318 aa, synthetic construct
DKGDGWILGEPSVVSSQILNPNETGTFSQSLTKSKEVSINVNFSVGFTSEFIQASVEYGFGITIGEQNTIERSVSTTAGPNEYVYYKVYATYRKYQAIRISHGNISDDGSIYKLTGIWLSKTSADSLGNIDQGSLIETGERCVLTVPSTDIEKEILDLAAATERLNLTDALNSNPAGNLYDWRSSNSYPWTQKLNLHLTITATGQKYRILASKIVDFNIYSNNFNNLVKLEQSLGDGVKDHYVDIHAFVNQHLCGSHLVEALYLVCGDRGFYFNKPTGYGSSSRRAPQTGIVDECCFRSCDLRRLEMYCAPLKPAKSA

SEQ ID NO: 15 - protein, 275 aa, synthetic construct
MHHHHHHGDDDDKDKGDGWILGEPSVVSSQILNPNETGTFSQSLTKSKEVSINVNFSVGFTSEFIQASVEYGFGITIGEQNTIERSVSTTAGPNEYVYYKVYATYRKYQAIRISHGNISDDGSIYKLTGIWLSKTSADSLGNIDQGSLIETGERCVLTVPSTDIEKEILDLAAATERLNLTDALNSNPAGNLMFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 16 - protein, 472 aa, synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHAMFPAMPLSSLFVNGPRTLCGAELVDALQFVCGDRGFYFNKPTGYGSSSRRAPQTGIVDECCFRSCDLRRLEMYCAPLKPAKSA

(19) United States
(12) Patent Application Publication
(10) Pub. No.: US 2025/0205347 A1
(43) Pub. Date: Jun. 26, 2025

(54) METHODS FOR TREATING INFLAMMATORY CONDITIONS OF THE EYE WITH AN IGF-1R LIGAND CONJUGATED TO A DISEASE-MODIFYING AGENT

(71) Applicant: Lirum Therapeutics, Inc., New York, NY (US)

(72) Inventor: Matthew HOBERMAN, Demarest, NJ (US)

(21) Appl. No.: 18/876,858

(22) PCT Filed: Jun. 29, 2023
(86) PCT No.: PCT/US2023/069428

§ 371 Date: Dec. 19, 2024

Related U.S. Application Data
(60) Provisional application No. 63/367,238, filed on Jun. 29, 2022.

(51) Int. Cl. A61K 47/64

(52) U.S. Cl. CPC A61K 47/642

(57) ABSTRACT

The present invention provides methods for treating an inflammatory condition of the eye in a subject, comprising administering to the subject an effective amount of a conjugate that comprises an IGF-IR ligand, or portion or variant thereof, and a disease-modifying agent.

### CROSS REFERENCE TO RELATED APPLICATIONS

This application claims the benefit of U.S. Provisional Application No. 63/367,238, filed on Jun. 29, 2022.

### FIELD

The presently disclosed subject matter relates generally to methods for treating inflammatory conditions of the eye in particular by administering to the subject an insulin-like growth factor 1 receptor (IGF-1R) ligand conjugated to a disease-modifying agent.

### REFERENCE TO A SEQUENCE LISTING

The Sequence Listing written in file name 597948SEQLIST.xml is 17 kilobytes, was created on Jun. 29, 2023, and is hereby incorporated by reference.

### BACKGROUND

Inflammatory conditions of the eye encompass a wide range of conditions, ranging from easily treatable to severe sight-threatening emergencies that can lead to permanent vision loss. Inflammation can occur in response to infection, allergies, irritation, injury, or trauma to the eye or surrounding tissues. In addition, many systemic autoimmune diseases have ocular manifestations.

One example is thyroid eye disease (TED), an autoimmune disease characterized by progressive inflammation and damage to tissues around the eye. TED occurs in approximately 40% of Graves' disease patients and is estimated to affect 16 in 100,000 women and 2.9 in 100,000 men, with men more often having severe disease. Pathological changes involve extraocular muscle enlargement and orbital fat expansion, leading to proptosis, which causes orbital congestion, periorbital edema, and other, sometimes severe, sight-threatening symptoms. Other manifestations include strabismus, diplopia, restricted ocular motility, eyelid retraction, pain, distorted vision, facial disfigurement, and diminished quality of life.

There remains a need in the art for improved methods of treating TED, as well as other inflammatory conditions of the eye.

### BRIEF SUMMARY

In accordance with the description, this application describes methods of treatment with an IGF-1R ligand conjugated to a disease-modifying agent.

In one embodiment, the present application provides a method for treating an inflammatory condition of the eye in a subject in need thereof comprising administering to the subject an effective amount of a conjugate, wherein said conjugate comprises an IGF-1R ligand and a disease-modifying agent.

In some embodiments, the inflammatory condition of the eye is TED, uveitis, scleritis, keratitis, or conjunctivitis.

In some embodiments, the inflammatory condition of the eye is an orbital inflammatory disease. In some embodiments, the orbital inflammatory disease is idiopathic orbital inflammation, orbital inflammatory pseudotumor, orbital myositis, inflammatory orbital cellulitis, optic perineuritis, periscleritis, diffuse orbital inflammation, orbital apicitis, or sclerosing orbital inflammation.

In some embodiments, the inflammatory condition of the eye is TED.

In some embodiments, the TED is acute TED, inactive TED, chronic TED, moderate-to-severe TED, or sight-threatening (very severe) TED.

In some embodiments, treatment of TED with the conjugate of the present invention results in a reduction in proptosis in said subject. In a specific embodiment, the reduction in proptosis is reduced by at least 2 mm, at least 3 mm, or at least 4 mm. In another embodiment, the reduction in proptosis is assessed by exophthalmometer or orbital imaging. In a specific embodiment, the orbital imaging is computerized tomography scan or magnetic resonance imaging. In some embodiments, the reduction in proptosis is associated with a reduction in extraocular muscle volume and/or orbital fat volume.

In some embodiments, treatment of TED with the conjugate of the present invention results in a reduction in the clinical activity score (CAS) in said subject. In a specific embodiment, the CAS is reduced by at least 2 points or at least 3 points. In another specific embodiment, the CAS is reduced to 1 or reduced to 0.

In some embodiments, treatment of TED with the conjugate of the present invention results in a reduction in the severity of diplopia in said subject. In another embodiment, the reduction in the severity of diplopia is measured by the Gorman subjective diplopia score. In some embodiments, the diplopia is constant diplopia, intermittent diplopia, or inconstant diplopia. In some embodiments, the treatment results in complete resolution of diplopia. In another embodiment, the severity of diplopia in the subject is reduced by at least one grade.

In some embodiments, treatment of TED with the conjugate of the present invention results in an improvement in the quality of life of said subject. In another embodiment, the improvement in the quality of life is measured by the Graves' Ophthalmopathy Quality of Life (GO-QoL) assessment scale or the Visual Functioning subscale or the Appearance subscale of the GO-QoL. In a specific embodiment, the quality of life is improved by at least 8 points on the GO-QoL scale or the Visual Functioning subscale of the GO-QoL or the Appearance subscale of the GO-QoL.

In some embodiments, the treatment results in an improvement in optic neuropathy.

In some embodiments, the treatment results in a reduction in retro-orbital edema.

In some embodiments, the treatment results in an improvement in monocular ductions. In a specific embodiment, the improvement in monocular ductions is measured by the light reflex test. In another specific embodiment, the improvement in monocular ductions is of at least 10 degrees.

In some embodiments, the treatment results in an alteration or impairment in the function of IGF-1R-expressing cells in said subject. In other embodiments, the treatment results in a reduction in the number of IGF-1R-expressing cells in the subject. In a specific embodiment, the reduction is caused by killing of IGF-1R-expressing cells. Without being bound by theory, it is believed that simple inhibition of the IGF-1R pathway is insufficient as a fully curative treatment due to signaling by other, potentially redundant, pathways that can continue to act as disease effectors or by compensatory mechanisms or pathways that are upregulated and can lead to adaptive resistance. Thus, the targeted delivery of an agent that is disease-modifying, including one that results in the killing of IGF-1R-expressing cells, may provide benefit beyond (including being preferential over) simple IGF-1R pathway inhibition, as it bypasses the effects of all such potentially redundant and/or compensatory pathways.

In another specific embodiment, the IGF-1R-expressing cells are orbital fibroblasts (OFs). In another embodiment, the treatment results in reduction or inhibition of hyaluronan synthesis in the retro-ocular space of said subject. In another embodiment, the treatment results in reduction or inhibition of adipogenesis in the retro-ocular space of said subject. In another embodiment, the treatment results in a reduction in the level of IL-6, IL-16, and/or RANTES in the serum of said subject.

In another specific embodiment, the IGF-IR-expressing cells are fibrocytes, B lymphocytes, and/or T lymphocytes.

In another embodiment, the IGF-1R is overexpressed by retro-ocular space cells in said subject relative to retro-ocular space cells from a healthy subject or a subject that has not been diagnosed with the inflammatory condition of the eye. In another embodiment, the frequency of cells expressing IGF-1R in said subject is increased relative to cells from a healthy subject or a subject that has not been diagnosed with the inflammatory condition of the eye. In another embodiment, the frequency of IGF-1R-expressing cells is measured by flow cytometry or immunohistochemistry.

In some embodiments, the treatment results in disease modification in said subject.

In some embodiments, the conjugate comprises wildtype insulin-like growth factor 1 (IGF-1) (SEQ ID NO:3), wildtype insulin (SEQ ID NO:10 and SEQ ID NO: 11), or wildtype insulin-like growth factor 2 (IGF-2) (SEQ ID NO:12). In some embodiments, the conjugate comprises a variant of wildtype IGF-1 (SEQ ID NO:3), a variant of wildtype insulin (SEQ ID NO: 10 and SEQ ID NO:11), or a variant of wildtype IGF-2 (SEQ ID NO:12). In some embodiments, the variant of wildtype IGF1 is at least 90% identical to IGF-1 (SEQ ID NO:3), the variant of wildtype insulin is at least 90% identical to insulin (SEQ ID NO:10 and SEQ ID NO:11), or the variant of wildtype IGF-2 is at least 90% identical to IGF-2 (SEQ ID NO:12).

In some embodiments, the conjugate comprises a variant of IGF-1 that has reduced binding affinity for IGF binding proteins (IGFBPs) as compared to wildtype IGF-1 (SEQ ID NO:3)) or a variant of IGF-2 that has reduced binding affinity for IGF binding proteins as compared to wildtype IGF-2 (SEQ ID NO:12). In some embodiments, the variant of IGF-1 has increased affinity for the IGF-1R than wildtype IGF-1 (SEQ ID NO:3) or the variant of IGF-2 has increased affinity for the IGF-1R than wildtype IGF-2 (SEQ ID NO: 12). In a specific embodiment, the conjugate comprises 765IGF (SEQ ID NO:2), IGF132 (SEQ ID NO:4), long-R3-IGF-1 (SEQ ID NO:5), R3-IGF-1 (SEQ ID NO:6), des (1-3)-IGF-1 (SEQ ID NO:7), long-IGF-1 (SEQ ID NO:8), or long-G3-IGF-1 (SEQ ID NO: 9).

In some embodiments, the IGF-1R ligand, or portion or variant thereof, comprises a leader sequence. In a specific embodiment, the leader sequence comprises SEQ ID NO:1.

In a specific embodiment, the conjugate comprises 765IGF (SEQ ID NO:2).

In some embodiments, the disease-modifying agent of the conjugate comprises a cytotoxic agent.

In some embodiments, the cytotoxic agent comprises a chemotherapeutic agent. In a specific embodiment, the chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine. In another specific embodiment, the chemotherapeutic agent is methotrexate.

In some embodiments, the cytotoxic agent comprises a toxin. In a specific embodiment, the toxin is Clostridium perfringens enterotoxin, diphtheria toxin, ricin A chain, deglycosylated ricin A chain, Pseudomonas exotoxin, A chain toxins, ribosome inactivating proteins, a-sarcin, aspergillin, abrin, restrictocin, bacterial endotoxin, the lipid A moiety of bacterial endotoxin, cholera toxin, or a ribonuclease. In a specific embodiment, the toxin comprises Clostridium perfringens enterotoxin, or a portion of variant thereof. In a specific embodiment, the conjugate comprises SEQ ID NO:14 or SEQ ID NO:15. In other specific embodiments, the toxin comprises diphtheria toxin, or a portion or variant thereof. In a specific embodiment, the conjugate comprises SEQ ID NO:13 or SEQ ID NO:16.

In other embodiments, the disease-modifying agent of the conjugate of the present invention is selected from: glucocorticoids (dexamethasone); corticosteroids (methylprednisolone, prednisone, triamcinolone acetonide); a TSHR inhibitor; mycophenolate mofetil; simvastatin; metformin; phenformin; cyclosporin; rapamycin or other mTOR inhibitors; or azathioprine.

In some embodiments, the subject with TED has Grave's hyperthyroidism and/or has a CAS of at least 3 or at least 4.

In some embodiments, the subject having an inflammatory condition of the eye treated in accordance with the methods described herein has not previously received treatment for said inflammatory condition of the eye, has previously received treatment for said inflammatory condition of the eye, has relapsed from previous treatment for said inflammatory condition of the eye, or was refractory to previous treatment for said inflammatory condition of the eye.

In some embodiments, the previous treatment comprised administration of an IGF-1R inhibitor to said subject.

In a specific embodiment, the IGF-1R inhibitor was an antibody. In another specific embodiment, the antibody is teprotumumab, ganitumab, figitumumab, MEDI-573, cixutumumab, dalotuzumab, robatumumab, AVE1642, BIIB022, xentuzumab, istiratumab, lonigutamab, VB421, VRDN-001, VRDN-002, or VRDN-003.

In a specific embodiment, the previous treatment comprised administration of an antibody or antibody fragment targeting the neonatal Fc receptor (FcRn). In a specific embodiment, the antibody or antibody fragment is batoclimab, IMVT-1402, or efgartigimod.

In a specific embodiment, the previous treatment comprised administration of an antibody targeting IL-6. In a specific embodiment, the antibody is TOUR006.

In a specific embodiment, the IGF-1R inhibitor was a small molecule. In another specific embodiment, the small molecule is linsitinib, picropodophyllin, AXL1717, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, or A-928605.

In some embodiments, the IGF-1R inhibitor was a radioimmunoconjugate. In a specific embodiment, the radioimmunoconjugate was FPI-1434.

In some embodiments, the IGF-1R inhibitor was an antibody-drug conjugate. In a specific embodiment, the antibody-drug conjugate was W0101.

In some embodiments, the conjugate is administered in combination with one or more other therapies. In a specific embodiment, the one or more other therapies comprises one or more of the following: glucocorticoids (dexamethasone); corticosteroids (methylprednisolone, prednisone, triamcinolone acetonide); rituximab or other anti-CD20 antibodies; tocilizumab, TOUR006, or other anti-IL-6 antibodies; selenium; infliximab, adalimumab, or other anti-tumor necrosis factor (TNF) a antibodies; a thyroid-stimulating hormone receptor (TSHR) inhibitor; orbital radiotherapy; a vascular growth factor (VEGF) inhibitor; mycophenolate mofetil; iscalimab or other anti-CD40 antibodies; IMVT-1401, batoclimab, efgartigimod, or other neonatal fragment crystallizable (Fc) receptor (FcRN) inhibitors; simvastatin; metformin; phenformin; cyclosporin; azathioprine; methotrexate; rapamycin or other mammalian target of rapamycin (mTOR) inhibitors; and/or intravenous immunoglobulin treatment.

In some embodiments, the conjugate is administered at a dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.

In some embodiments, the conjugate is administered at a dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.

In some embodiments, the conjugate is administered at a dose that is the maximum tolerated dose (MTD).

In some embodiments, the conjugate is administered daily, every other day, every three days, every four days, every five days, every six days, once per week, once every two weeks, once every three weeks, once every four weeks, once per month, every two months, or every three months.

In some embodiments, the conjugate is administered intravenously, subcutaneously, via intravitreal injection, via intra-orbital injection, or via eye dropper.

In some embodiments, the method of the present invention does not cause unacceptable hyperglycemia in the subject.

In a specific embodiment, provided herein is a method for treating TED in a subject in need thereof by administering an effective amount of a conjugate, wherein said conjugate comprises 765IGF (SEQ ID NO:2) and methotrexate.

These and other embodiments are described fully below.

### DETAILED DESCRIPTION

The subject matter described herein relates to methods of treating inflammatory conditions of the eye using a targeted therapy directed to IGF-1R, that comprises an IGF-1R ligand or variant thereof in combination with a disease-modifying agent.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents and other references mentioned herein are incorporated by reference into their entirety. In the event that one or more of the incorporated literature, patents, and similar materials differs from or contradicts this application, including but not limited to defined terms, term usage, described techniques, or the like, this application controls.

### I. Definitions

As used herein, a patient or subject and the like is any mammal suffering from an inflammatory condition of the eye. As used herein, the term “mammal” includes, but is not limited to, humans, mice, rats, guinea pigs, monkeys, dogs, cats, horses, cows, pigs, and sheep.

As used herein, the term “conjugate” refers to a molecule comprising an IGF-1R ligand, or portion or variant thereof, and a disease-modifying agent.

As used herein, the term “cytotoxic agent” refers to any agent capable of preventing, delaying, reducing and/or reversing the activity, severity, and/or progression of the disease when treated in accordance with the methods described herein. Any suitable cytotoxic agent that results in cell killing can be used in the conjugate and in the method of treating an inflammatory condition of the eye.

As used herein, the term “residue” or “residue of” a chemical moiety or compound refers to a chemical moiety or compound that is bound to a molecule, whereby through the binding, at least one covalent bond has replaced at least one atom of the original chemical moiety or compound, resulting in a residue of the chemical moiety or compound in the molecule.

As used herein, a subject is “refractory” to prior treatment if the subject has failed to achieve a response to a therapy such that the therapy is determined to not be therapeutically effective, such as: failure to reach clinical endpoint, including any of response, extended duration of response, extended disease-free survival, relapse-free survival, and progression-free survival.

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.1% of the specified amount.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Definitions of additional terms may be set forth below.

### II. Compositions

### Agents for Treatment

This application provides methods for treating an inflammatory condition of the eye in a subject in need thereof comprising administering to the subject an effective amount of a conjugate, wherein said conjugate comprises an IGF-1R ligand, or portion or variant thereof, and a disease-modifying agent.

In this or any of the embodiments of the present invention, the conjugate can comprise a chemical conjugate in which the IGF-1R ligand and the disease-modifying agent are chemically linked together, either directly or through a chemical linker. In other embodiments, the conjugate is a genetic recombinant in which the conjugate is expressed as a single polypeptide. When the conjugate is a recombinant conjugate, the translated conjugate preferably comprises a toxin, or portion or variant thereof, linked via a peptide bond to the IGF-1R ligand.

In certain embodiments, the conjugate is a fusion protein described in U.S. Pat. No. 9,675,671, which is hereby incorporated by reference in its entirety.

### IGF-1R Ligands

The IGF-1R is a heterotetramer consisting of two extracellular ligand-binding a subunits and two transmembrane β subunits with kinase activity that mediate signal transduction. The native ligands to the IGF-IR are IGF-1, IGF-2, and insulin. The IGF-1R has the highest affinity for IGF-1, followed by IGF-2, and can bind to insulin with 50-to 100-fold lower affinity. IGF-1R can also form hybrid receptors by dimerization with the insulin receptor. See Hakuno et al. J Mol Endocrinol. 61(1): T69-T86 (2018).

In certain embodiments, the IGF-1R ligand in the conjugate of the present invention comprises wildtype IGF-1 (SEQ ID NO:3), wildtype insulin (SEQ ID NO: 10 and SEQ ID NO:11; mature insulin consists of two chains connected by disulfide bonds, chain A, corresponding to SEQ ID NO: 10, and chain B, corresponding to SEQ ID NO:11, hence the recitation of two SEQ ID NOs), or wildtype IGF-2 (SEQ ID NO:12). In other embodiments, the IGF-1R ligand in the conjugate comprises a variant of wildtype IGF-1 (SEQ ID NO:3), a variant of wildtype insulin (SEQ ID NO:10 and SEQ ID NO:11), or a variant of wildtype IGF-2 (SEQ ID NO:12). In a specific embodiment, the variant of wildtype IGF-1 is at least 70%, 75%, 80%, 85%, 90%, 95%, or 99% identical to IGF-1 (SEQ ID NO:3), said variant of wildtype insulin is at least 70%, 75%, 80%, 85%, 90%, 95%, or 99% identical to insulin (SEQ ID NO:10 and SEQ ID NO:11), or said variant of wildtype IGF-2 is at least 70%, 75%, 80%, 85%, 90%, 95%, or 99% identical to IGF-2 (SEQ ID NO:12).

In another specific embodiment, the IGF-1R ligand in the conjugate comprises a variant of IGF-1 that has reduced binding affinity for IGFBPs as compared to wildtype IGF-1 (SEQ ID NO:3) or a variant of IGF-2 that has reduced binding affinity for IGFBPs as compared to wildtype IGF-2 (SEQ ID NO:12). IGFBPs belong to a family of at least six proteins that bind to IGF-1 and IGF-2 with high affinity. IGFBPs bind to the majority of IGFs in circulation, increasing their half-life, regulating their bioavailability, and generally inhibit their ability to bind to the IGF receptors. See Baxter, Am J Physiol Endocrinol Metab., 278(6): E967-76 (2000) and Allard et al. Front Endocrinol (Lausanne). 9;9:117 (2018). Thus, variants of IGF-1 or IGF-2 that have reduced binding to IGFBPs have greater bioactivity in vivo.

IGF-1 variants with reduced binding affinity for IGFBPs are known in the art and include IGF132 (disclosed in U.S. Pat. No. 4,876,242), in which the first 17 amino acids of the B chain of insulin (SEQ ID NO:11) replace the first 16 amino acids of human IGF-1 (SEQ ID NO:3); R3-IGF-1 (SEQ ID NO:6), in which glutamic acid in position 3 of the native human IGF-1 (SEQ ID NO:3) is substituted by arginine; and des(1-3) IGF-1 (SEQ ID NO:7), which lacks the first three amino acids of human IGF-1 (SEQ ID NO: 3). R3-IGF-1 and des(1-3) IGF-1 are described in Francis et al., J Mol Endocrinol. 8(3): 213-23 (1992). In some embodiments, the conjugate comprises IGF132 (SEQ ID NO: 4), R3-IGF-1 (SEQ ID NO:6), or des(1-3)-IGF-1 (SEQ ID NO:7).

In another embodiment, the variant of IGF-1 has higher affinity for the IGF-1R than wildtype IGF-1 (SEQ ID NO:3), or the variant of IGF-2 has higher affinity for the IGF-1R than wildtype IGF-2 (SEQ ID NO:12).

In another embodiment, the IGF-1R ligand, or portion or variant thereof, comprises a leader sequence. Leader sequences can incorporate tags, such as polyhistidine tags, to facilitate protein purification, as well as provide sites for conjugation of disease-modifying agents. In a specific embodiment, the leader sequence comprises SEQ ID NO:1.

In some embodiments, the IGF-1R ligand in the conjugate comprises 765IGF (SEQ ID NO:2), long-R3-IGF-1 (SEQ ID NO:5), long-IGF-1 (SEQ ID NO:8), or long-G3-IGF-1 (SEQ ID NO:9). 765IGF, long-R3-IGF-1, long-IGF-1, and long-G3-IGF-1 have N-terminal leader sequences that facilitate protein purification and provide sites for conjugation of disease-modifying agents, as described above. 765IGF (SEQ ID NO:2) comprises SEQ ID NO:1 followed by R3-IGF-1 (SEQ ID NO:6); long-R3-IGF-1 (SEQ ID NO: 5) comprises the first 11 amino acids of methionyl porcine growth hormone, followed by a Val-Asn dipeptide, followed by R3-IGF-1 (SEQ ID NO:6); long-IGF-1 (SEQ ID NO: 8) comprises the first 11 amino acids of methionyl porcine growth hormone, followed by a Val-Asn dipeptide, followed by human IGF-1 (SEQ ID NO:3); and long-G3-IGF-1 comprises the first 11 amino acids of methionyl porcine growth hormone, followed by a Val-Asn dipeptide, followed by a variant of human IGF-1 in which glutamic acid in position 3 of the native human IGF-1 (SEQ ID NO:3) is substituted by glycine. In some embodiments, the conjugate comprises 765IGF (SEQ ID NO:2).

### Disease-Modifying Agents

As used herein, the term disease-modifying agent refers to any agent capable of preventing, delaying, reducing and/or reversing the activity, severity, and/or progression of the disease when treated in accordance with the methods described in the present invention.

In a specific embodiment, the disease-modifying agent of the conjugate is a cytotoxic agent. Any suitable cytotoxic agent that results in cell killing can be used in the conjugate of the invention and in the method of treating an inflammatory condition of the eye.

In a specific embodiment, the cytotoxic agent is a chemotherapeutic agent Any suitable chemotherapeutic agent that results in cell killing can be used in the conjugate of the invention and in the method of treating an inflammatory condition of the eye. For instance, in particular embodiments, the chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine. In a specific embodiment, the chemotherapeutic agent is methotrexate.

In certain embodiments, a conjugate comprises more than one cytotoxic agent bound to the IGF-1R ligand. In certain aspects, the conjugate can comprise one to 12 cytotoxic agents, or 6 to 10 cytotoxic agents, or about 8 cytotoxic agents. In certain aspects, the conjugate can comprise one to twelve covalently bound cytotoxic agents, or 6 to 10 covalently bound cytotoxic agents, or about 8 covalently bound cytotoxic agents. In certain aspects, the cytotoxic agent(s) are covalently bound to any available position on the IGF-1R ligand. In certain aspects, the cytotoxic agent(s) are covalently bound to any available lysine residue. In certain aspects, the cytotoxic agent(s) are covalently bound to any available lysine in the leader sequence when present.

In other embodiments, the cytotoxic agent is a toxin. Any suitable toxin that results in cell killing can be used in the conjugate of the present invention and in the method of treating an inflammatory condition of the eye. Toxins can be derived from plants, fungus, or bacteria. For instance, in particular embodiments, the toxin is Clostridium perfringens enterotoxin, diphtheria toxin, ricin A chain, deglycosylated ricin A chain, Pseudomonas exotoxin, A chain toxins, ribosome inactivating proteins, α-sarcin, aspergillin, abrin, restrictocin, bacterial endotoxin, the lipid A moiety of bacterial endotoxin, cholera toxin, or a ribonuclease. In a specific embodiment, the toxin comprises Clostridium perfringens enterotoxin, or a portion of variant thereof. In a specific embodiment, the conjugate comprises SEQ ID NO:14 or SEQ ID NO:15. In other specific embodiments, the toxin comprises diphtheria toxin, or a portion or variant thereof. In a specific embodiment, the conjugate comprises SEQ ID NO:13 or SEQ ID NO:16.

In another specific embodiment, the disease-modifying agent of the conjugate of the present invention is selected from: glucocorticoids (dexamethasone); corticosteroids (methylprednisolone, prednisone, triamcinolone acetonide); a TSHR inhibitor; mycophenolate mofetil; simvastatin; metformin; phenformin; cyclosporin; rapamycin or other mTOR inhibitors; or azathioprine.

Table 1 provides a listing of certain sequences referenced herein.

TABLE 1
Description of the Sequences

| Description | Sequences | SEQ ID NO |
|---|---|---|
| Leader | MVKGKHHHHHHNGKGKSK | 1 |
| sequence | | |
| 765IGF | MVKGKHHHHHHNGKGKSKGPRTLCGAELVDALQF | 2 |
| | VCGDRGFYFNKPTGYGSSSRRAPQTGIVDECCFRSC | |
| | DLRRLEMYCAPLKPAKSA | |
| human IGF-1 | GPETLCGAELVDALQFVCGDRGFYFNKPTGYGSSS | 3 |
| | RRAPQTGIVDECCFRSCDLRRLEMYCAPLKPAKSA | |
| IGF132 | FVNQHLCGSHLVEALYLVCGDRGFYFNKPTGYGSS | 4 |
| | SRRAPQTGIVDECCFRSCDLRRLEMYCAPLKPAKSA | |
| Long-R3-IGF-1 | MFPAMPLSSLFVNGPRTLCGAELVDALQFVCGDRG | 5 |
| | FYFNKPTGYGSSSRRAPQTGIVDECCFRSCDLRRLE | |
| | MYCAPLKPAKSA | |
| R3-IGF-1 | GPRTLCGAELVDALQFVCGDRGFYFNKPTGYGSSS | 6 |
| | RRAPQTGIVDECCFRSCDLRRLEMYCAPLKPAKSA | |
| des(1-3)IGF-1 | TLCGAELVDALQFVCGDRGFYFNKPTGYGSSSRRA | 7 |
| | PQTGIVDECCFRSCDLRRLEMYCAPLKPAKSA | |
| Long-IGF-1 | MFPAMPLSSLFVNGPETLCGAELVDALQFVCGDRG | 8 |
| | FYFNKPTGYGSSSRRAPQTGIVDECCFRSCDLRRLE | |
| | MYCAPLKPAKSA | |
| Long-G3-IGF-1 | MFPAMPLSSLFVNGPGTLCGAELVDALQFVCGDRG | 9 |
| | FYFNKPTGYGSSSRRAPQTGIVDECCFRSCDLRRLE | |
| | MYCAPLKPAKSA | |
| Human insulin | GIVEQCCTSICSLYQLENYCN | 10 |
| A-chain | | |
| Human insulin | FVNQHLCGSHLVEALYLVCGERGFFYTPKT | 11 |
| B-chain | | |
| Human IGF-2 | AYRPSETLCGGELVDTLQFVCGDRGFYFSRPASRVS | 12 |
| | RRSRGIVEECCFRSCDLALLETYCATPAKSE | |
| Diphtheria | GADDVVDSSKSFVMENFSSYHGTKPGYVDSIQKGI | 13 |
| toxin-IGF132 | QKPKSGTQGNYDDDWKGFYSTDNKYDAAGYSVD | |
| fusion | NENPLSGKAGGVVKVTYPGLTKVLALKVDNAETIK | |
| | KELGLSLTEPLMEQVGTEEFIKRFGDGASRVVLSLP | |
| | FAEGSSSVEYINNWEQAKALSVELEINFETRGKRGQ | |
| | DAMYEYMAQACAGNRVRRSVGSSLSCINLDWDVI | |
| | RDKTKTKIESLKEHGPIKNKMSESPNKTVSEEKAKQ | |
| | YLEEFHQTALEHPELSELKTVTGTNPVFAGANYAA | |
| | WAVNVAQVIDSETADNLEKTTAALSILPGIGSVMGI | |
| | ADGAVHHNTEEIVAQSIALSSLMVAQAIPLVGELVD | |
| | IGFAAYNFVESIINLFQVVHNSYNRPAYSPGHKTQPF | |
| | HAFVNQHLCGSHLVEALYLVCGDRGFYFNKPTGY | |
| | GSSSRRAPQTGIVDECCFRSCDLRRLEMYCAPLKPA | |
| | KSA | |
| Clostridium | DKGDGWILGEPSVVSSQILNPNETGTFSQSLTKSKE | 14 |
| perfringens | VSINVNFSVGFTSEFIQASVEYGFGITIGEQNTIERSV | |
| enterotoxin- | STTAGPNEYVYYKVYATYRKYQAIRISHGNISDDGS | |
| IGF132 fusion | IYKLTGIWLSKTSADSLGNIDQGSLIETGERCVLTVP | |
| | STDIEKEILDLAAATERLNLTDALNSNPAGNLYDWR | |
| | SSNSYPWTQKLNLHLTITATGQKYRILASKIVDFNIY | |
| | SNNFNNLVKLEQSLGDGVKDHYVDIHAFVNQHLC | |
| | GSHLVEALYLVCGDRGFYFNKPTGYGSSSRRAPQT | |
| | GIVDECCFRSCDLRRLEMYCAPLKPAKSA | |
| Clostridium | MHHHHHHGDDDDKDKGDGWILGEPSVVSSQILNP | 15 |
| perfringens | NETGTFSQSLTKSKEVSINVNFSVGFTSEFIQASVEY | |
| enterotoxin- | GFGITIGEQNTIERSVSTTAGPNEYVYYKVYATYRK | |
| long-R3-IGF-1 | YQAIRISHGNISDDGSIYKLTGIWLSKTSADSLGNID | |
| fusion | QGSLIETGERCVLTVPSTDIEKEILDLAAATERLNLT | |
| | DALNSNPAGNLMFPAMPLSSLFVNGPRTLCGAELV | |
| | DALQFVCGDRGFYFNKPTGYGSSSRRAPQTGIVDEC | |
| | CFRSCDLRRLEMYCAPLKPAKSA | |
| Diphtheria | MGADDVVDSSKSFVMENFSSYHGTKPGYVDSIQKG | 16 |
| toxin-long-R3- | IQKPKSGTQGNYDDDWKGFYSTDNKYDAAGYSVD | |
| IGF-1 fusion | NENPLSGKAGGVVKVTYPGLTKVLALKVDNAETIK | |
| | KELGLSLTEPLMEQVGTEEFIKRFGDGASRVVLSLP | |
| | FAEGSSSVEYINNWEQAKALSVELEINFETRGKRGQ | |
| | DAMYEYMAQACAGNRVRRSVGSSLSCINLDWDVI | |
| | RDKTKTKIESLKEHGPIKNKMSESPNKTVSEEKAKQ | |
| | YLEEFHQTALEHPELSELKTVTGTNPVFAGANYAA | |
| | WAVNVAQVIDSETADNLEKTTAALSILPGIGSVMGI | |
| | ADGAVHHNTEEIVAQSIALSSLMVAQAIPLVGELVD | |
| | IGFAAYNFVESIINLFQVVHNSYNRPAYSPGHKTHA | |
| | MFPAMPLSSLFVNGPRTLCGAELVDALQFVCGDRG | |
| | FYFNKPTGYGSSSRRAPQTGIVDECCFRSCDLRRLE | |
| | MYCAPLKPAKSA | |

### Pharmaceutical Compositions

The conjugates of the present invention can be formulated in pharmaceutical compositions for use in the methods described herein. In some embodiments, the pharmaceutical composition comprises an effective amount of a conjugate of the invention and a pharmaceutically acceptable carrier or vehicle. Such pharmaceutical compositions can be formulated to be suitable for administration to a subject and can be in any form that allows for the composition to be administered to a subject.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of condition the subject has, the use of the composition as part of a multi-drug regimen, the particular form of the composition of the invention, and the manner of administration. The pharmaceutical compositions comprise an effective amount of a composition of the invention such that a suitable dosage will be obtained.

The term “carrier” refers to a diluent, adjuvant or excipient, with which a composition of the invention is administered. Any auxiliary, stabilizing, thickening, lubricating and coloring agents can be used. In one embodiment, when administered to a subject, the compositions of the invention and pharmaceutically acceptable carriers are sterile. Water may be a carrier when the composition of the invention is administered intravenously. Saline solutions and aqueous dextrose and glycerol solutions can also be employed as liquid carriers, particularly for injectable solutions. The present compositions, if desired, can also contain minor amounts of pH buffering agents.

The liquid compositions of the invention, whether they are solutions, suspensions, or other like form, can also include one or more of the following: sterile diluents such as water for injection, saline solution, physiological saline, Ringer's solution, isotonic sodium chloride, fixed oils such as synthetic mono or digylcerides which can serve as the solvent or suspending medium, polyethylene glycols, glycerin, cyclodextrin, propylene glycol, or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; agents for the adjustment of pH such as hydrochloric acid; and agents for the adjustment of tonicity such as sodium chloride or dextrose. A parenteral composition can be enclosed in an ampoule, a disposable syringe, or a multiple-dose vial made of glass, plastic or other material. In some embodiments, physiological saline is an adjuvant. An injectable composition may be sterile.

The present compositions can take the form of solutions, suspensions, tablets, pills, pellets, capsules, capsules containing liquids, powders, sustained-release formulations, suppositories, emulsions, aerosols, sprays, suspensions, or any other form suitable for use. Examples of suitable pharmaceutical carriers are described in Remington's Pharmaceutical Sciences by E.W. Martin.

In some embodiments, the compositions of the invention are formulated in accordance with routine procedures as a pharmaceutical composition adapted for intravenous administration to human subjects. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where a composition of the invention is to be administered by infusion, it can be dispensed, for example, with an infusion bottle containing sterile pharmaceutical grade water or saline. Where the composition of the invention is administered by injection, an ampoule of sterile water for injection or saline can be provided so that the ingredients can be mixed prior to administration.

The pharmaceutical compositions can be prepared using methodology well known in the pharmaceutical art. For example, a composition intended to be administered by injection can be prepared by combining a composition of the invention with water so as to form a solution. A surfactant can be added to facilitate the formation of a homogeneous solution or suspension. Surfactants are complexes that can non-covalently interact with a composition of the invention so as to facilitate dissolution or homogeneous suspension of the composition of the invention in the aqueous delivery system.

### III. Methods

### Methods for Producing IGF-1R Conjugates

Methods for producing conjugates of the present invention are known in the art.

The nucleotide sequences encoding the IGF-1R ligands can be produced by standard recombinant DNA techniques or by protein synthetic techniques, cloned into an appropriate expression vector using standard molecular biology techniques, expressed in bacterial, insect, or mammalian cells, and purified by any method known in the art for purification of a protein.

Conjugates of the present invention comprising an IGF-1R ligand and a chemotherapeutic agent can be made by standard chemistry and protein conjugation techniques and are described in U.S. Pat. Nos. 7,811,982; 9,675,671; and 9,801,923.

Conjugates of the present invention comprising an IGF-1R ligand and a toxin can be made as fusion proteins by standard recombinant DNA techniques and are described in U.S. Pat. No. 8,017,102.

### Methods of Treatment

Provided herein are methods of treating inflammatory conditions of the eye using a conjugate that comprises an IGF-1R ligand, or portion or variant thereof, and a disease-modifying agent.

In some embodiments, the inflammatory condition of the eye is TED, uveitis, scleritis, keratitis, or conjunctivitis.

In some embodiments the inflammatory condition of the eye is an orbital inflammatory disease. In certain embodiments, the orbital inflammatory disease is idiopathic orbital inflammation, orbital inflammatory pseudotumor, orbital myositis, inflammatory orbital cellulitis, optic perineuritis, periscleritis, diffuse orbital inflammation, orbital apicitis, or sclerosing orbital inflammation.

In a specific embodiment, the inflammatory condition of the eye is TED. TED may also be referred to as Grave's ophthalmopathy, thyroid associated ophthalmopathy, Graves' eye disease, Graves' orbitopathy, or thyroid inflammatory eye disease.

In certain embodiments, the subject matter described herein is directed to methods for treating inflammatory conditions of the eye in a subject, the method comprising administering to the subject a conjugate comprising an IGF-1R ligand, or portion or variant thereof, and a cytotoxic agent, wherein the IGF-1R ligand, or portion or variant thereof comprises SEQ ID NO:2, the cytotoxic agent is methotrexate, wherein the methotrexate is covalently bound to a lysine of SEQ ID NO: 2, and the inflammatory conditions of the eye is selected from the group consisting of thyroid eye disease, uveitis, scleritis, keratitis, conjunctivitis, and an orbital inflammatory disease. In a certain aspect of these embodiments, the conjugate is LX-101 (a conjugate as described above wherein the IGF-1R ligand is SEQ ID NO:2, the cytotoxic agent is methotrexate, and wherein at least 6 and up to 10, or at least 6 and up to 9, or at least 7 and up to 9, or at least 8 and up to 9 methotrexate residues are each covalently bound to a lysine residue of SEQ ID NO:2).

In certain embodiments, the number of methotrexate residues per conjugate is 6, 7, 8, 9 or 10. In certain embodiments, the average number of methotrexate residues per conjugate in a composition is 6, 7, 8, 9 or 10.

### Methods of Measuring Clinical Efficacy

As used herein, the terms “subject” and “patient” are used interchangeably.

In some embodiments, the method for treating TED in a subject comprising administering the conjugates of the present invention to a subject in need thereof results in a reduction in proptosis in the subject. Proptosis, also called exophthalmos, is protrusion of the eyeball, which causes bulging of the eye, and can affect one or both eyes. In TED, proptosis is caused by expansion of the orbital fat and muscles behind the eyes. See Khong et al., Br J Ophthalmol, 100(1): 142-50 (2016). In some embodiments, the reduction in proptosis is reduced by greater than 2 mm, for example, 2.2 mm, 2.4 mm, 2.5 mm, 2.6 mm. 2.8 mm, 3 mm, 3.2 mm, 3.4 mm, 3.5 mm, 3.6 mm, 3.8 mm, 4 mm, 4.1 mm, 4.2 mm, 4.3 mm, 4.4 mm, 4.5 mm, 4.6 mm, 4.7 mm, 4.8 mm, 4.9 mm, 5 mm or more than 5 mm. In a specific embodiment, proptosis is reduced by at least 2 mm, at least 3 mm, or at least 4 mm. In another embodiment, the reduction in proptosis is associated with a reduction in extraocular muscle volume and/or orbital fat volume in the subject. In another embodiment, the reduction in proptosis is assessed by exophthalmometer or orbital imaging. In a specific embodiment, the orbital imaging is computerized tomography scan or magnetic resonance imaging.

In some embodiments, the method for treating TED in a subject comprising administering the conjugates of the present invention to a subject in need thereof results in a reduction in the CAS in the subject. The CAS consists of seven components (spontaneous retrobulbar pain, pain on attempted upward or downward gaze, redness of eyelids, redness of conjunctiva, swelling of the caruncle or plica, swelling of eyelids, and chemosis), each of which is scored as 1 (present) or 0 (absent). The CAS is the sum of all items present and ranges from 0-7, where a score of 0 or 1 constitutes inactive disease, a score of 3 or more constitutes active disease, and a score of 7 constitutes severe active disease. See Bartalena et al. Eur J Endocrinol. 185(4): G43-G67 (2021). A change of 2 or more points is considered clinically meaningful. In some embodiments, the reduction in CAS is by 2 points or more, for example, by 3, 4, 5, 6, or 7 points. In one embodiment, the reduction in CAS is by 2 or more points. In another embodiment. it is by 3 or more points. In yet another embodiment, the reduction in CAS is by 4 or more points. In a specific embodiment, the CAS is reduced by at least 2 points or at least 3 points. In another specific embodiment, the CAS is reduced to 1 or reduced to 0.

In some embodiments, the method for treating TED in a subject comprising administering the conjugates of the present invention to a subject in need thereof results in a reduction in the severity of diplopia in the subject. In another embodiment, the reduction in diplopia is measured by the Gorman subjective diplopia score. Diplopia is the medical term for double vision and is assessed by the Gorman subjective diplopia score, which includes 4 categories: no diplopia (absent, score of 0), diplopia in primary position of gaze when the patient is tired or awakening (intermittent, score of 1), diplopia at extremes of gaze (inconstant, score of 2), and continuous diplopia in the primary or reading position (constant, score of 3). See Bartalena et al. Eur J Endocrinol, 158(3): 273-85 (2008). An improvement of one grade or more is considered clinically meaningful. In certain embodiments, the diplopia is constant diplopia, intermittent diplopia, or inconstant diplopia. In a specific embodiment, the treatment results in complete resolution of diplopia. In another specific embodiment, the severity of diplopia in the subject is reduced by at least one grade.

In some embodiments, the method for treating TED in a subject comprising administering the conjugates of the present invention to a subject in need thereof results in an improvement in the quality of life of the subject. Quality of life is measured by the GO-QoL assessment scale, which is a self-administered questionnaire consisting of two subscales with eight questions each: a Visual Functioning subscale, which assesses the impact of visual function on daily activities, and an Appearance subscale, which assesses the impact on self-perceived appearance. Each question is scored 0-2, and the total raw score is transformed to a 0-100 scale, where 0 represents the most negative impact on quality of life and 100 represents no impact on quality of life. Raw scores from both subscales are also combined and transformed to a 0-100 scale. See Terwee, et al. Br J Ophthalmol, 82(7): 773-9 (1998). A change of 8 or more points has been shown to be clinically meaningful. In a specific embodiment, the improvement in the quality of life is measured by the GO-QoL assessment scale or the Visual Functioning subscale or the Appearance subscale of the GO-QoL. In another specific embodiment, the quality of life is improved by at least 8 points on the GO-QoL assessment scale, the Visual Functioning subscale of the GO-QoL, or the Appearance subscale of the GO-QoL.

Optic neuropathy is a serious sight-threatening complication of TED that can result from compression or stretching of the optic nerve. In some embodiments, the method for treating TED in a subject comprising administering the conjugates of the present invention to a subject in need thereof results in an improvement in optic neuropathy.

In some embodiments, the method for treating TED in a subject comprising administering the conjugates of the present invention to a subject in need thereof results in a reduction in retro-orbital edema.

Restricted ocular motility resulting from swelling in the muscles that control eye movement is another complication in TED. In some embodiments, the method for treating TED in a subject comprising administering the conjugates of the present invention to a subject in need thereof results in an improvement in monocular ductions. In a specific embodiment, the improvement in monocular ductions is measured by the light reflex test. In another specific embodiment, the improvement in monocular ductions is of at least 10 degrees.

In some embodiments, the method for treating an inflammatory condition of the eye in a subject comprising administering the conjugates of the present invention to a subject in need thereof results in an alteration or impairment in the function of IGF-1R-expressing cells in said subject. As used herein, alteration or impairment refers to any changes to the endogenous function of said IGF-1R-expressing cells in the inflammatory condition of the eye.

In some embodiments, the method for treating the inflammatory condition of the eye in a subject comprising administering the conjugates of the present invention to a subject in need thereof results in a reduction in the number of IGF-1R-expressing cells in the subject. In a specific embodiment, the reduction is caused by killing of IGF-1R-expressing cells. Without being bound by theory, it is believed that simple inhibition of the IGF-1R pathway is insufficient as a fully curative treatment due to signaling by other, potentially redundant, pathways that can continue to act as disease effectors or by compensatory pathways that are upregulated and can lead to adaptive resistance. Thus, the targeted delivery of an agent that is disease-modifying, including one that results in the killing of IGF-1R-expressing cells, may provide benefit beyond (including being preferential over) simple IGF-1R pathway inhibition, as it bypasses the effect of all such potentially redundant and/or compensatory pathways.

In another specific embodiment, the IGF-IR-expressing cells are orbital fibroblasts (OFs). OFs are central to the pathophysiology of TED. Activated OFs can proliferate and differentiate into adipocytes and myofibroblasts and secrete extracellular matrix proteins such as hyaluronan, leading to soft tissue expansion, all of which contribute to the various clinical manifestations of TED. Hyaluronan is the salt of hyaluronic acid (HA). Hyaluronan and hyaluronic acid may be used interchangeably herein. Cytokines and inflammatory mediators produced by immune cells infiltrating the orbit (e.g., T cells, B cells, mast cells, and monocytes) can promote the differentiation of OFs into myofibroblasts and adipocytes, leading to scarring, tissue remodeling, and tissue expansion. Myofibroblasts are contractile cells that express smooth muscle actin (aSMA). In some embodiments, the treatment results in reduction or inhibition of hyaluronan synthesis in the retro-ocular space of the subject. In some embodiments, the treatment results in a reduction in the levels of hyaluronan or hyaluronic acid. In other embodiments, the treatment results in reduction or inhibition of adipogenesis in the retro-ocular space of the subject. In some embodiments, the treatment results in a reduction in the levels of IL-6, IL-16, and/or RANTES in the serum of the subject. In some embodiments, the treatment results in a reduction or inhibition of the differentiation of OFs into myofibroblasts.

In another specific embodiment, the IGF-1R-expressing cells are fibrocytes, B lymphocytes, and/or T lymphocytes. Fibrocytes are fibroblast-like progenitors derived from the bone marrow that circulate in the periphery and have both tissue remodeling and immune activation properties. Without being bound to theory, it is believed that fibrocytes can infiltrate the orbit, differentiate into OFs, and contribute to the pathophysiology of TED and potentially other inflammatory conditions of the eye. B lymphocytes produce pathogenic autoantibodies that can cause tissue damage and/or dysregulate cellular signaling. T lymphocytes can activate autoreactive B cells as well as OFs and can contribute to inflammation and tissue damage.

In some embodiments, the IGF-1R is overexpressed by cells in the retro-ocular space relative to those from a healthy subject or a subject that has not been diagnosed with an inflammatory condition of the eye. In some embodiments, the frequency of cells expressing IGF-1R in the subject is increased relative to cells from a healthy subject or a subject that has not been diagnosed with an inflammatory condition of the eye. In some embodiments, the frequency of IGF-1R-expressing cells is measured by flow cytometry or immunohistochemistry using techniques and cell surface markers that are known in the art.

In some embodiments, the method for treating an inflammatory condition of the eye in a subject in need thereof with the conjugates of the present invention results in disease modification in the subject. As used herein, disease modification can refer to any prevention, delay, reduction, and/or reversal in the activity, severity, and/or progression of the disease in a subject when treated in accordance with the methods described in the present invention. Disease modification can result from direct or indirect effects on the underlying pathophysiology of the disease as a result of treatment with the conjugates of the present invention.

### Combination Treatments

In some embodiments, the conjugate of the present invention is administered in combination with one or more other therapies. Any therapy that is used for the treatment of TED can be used in combination with the conjugate of the present invention, including: glucocorticoids (dexamethasone); corticosteroids (methylprednisolone, prednisone, triamcinolone acetonide); rituximab or other anti-CD20 antibodies; tocilizumab, TOUR006, or other anti-IL-6 antibodies; selenium; infliximab, adalimumab, or other anti-TNFα antibodies; a TSHR inhibitor; orbital radiotherapy; VEGF inhibitors; mycophenolate mofetil; iscalimab or other anti-CD40 antibodies; IMVT-1401, batoclimab, efgartigimod, or other FcRN inhibitors; simvastatin; metformin; phenformin; cyclosporin; azathioprine; methotrexate; rapamycin or mTOR inhibitors; and/or intravenous immunoglobulin treatment.

In some embodiments, the combination of agents disclosed herein allows one or more agents to be dosed at a lower dosage level than the dose at which said agent would have an effect when dosed as a single agent.

### Dosing and Administration

The agents of this invention may be dosed at any clinically relevant dose. By clinically relevant, it is meant that the dose of the agent has an effect in the subject.

In some embodiments, the conjugate of the present invention is administered at a dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, or 10.0 μEq/kg of body weight, where a μEq is equivalent to a μmol of chemotherapeutic agent groups conjugated to the IGF-1R ligand.

In some embodiments, the conjugate of the present invention is administered at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 uEq/kg of body weight, where a uEq is equivalent to a μmol of chemotherapeutic agent groups conjugated to the IGF-1R ligand.

In some embodiments, the conjugate of the present invention is administered at a dose of about 0.05, 0.10, 0.15. 0.20, 0.25, 0.30, 0.35, 0.40, 0.45, 0.50, 0.55, 0.60, 0.65, 0.70, 0.75, 0 80, 0.85, 0.90, 0.95, 1 0, 1 1, 1.2. 1.3, 1.4, 1.5, 1.6, 1.7, 1.8, 1.9, 2.0, 2 1, 2 2, 2.3, 2.4, 2.5, 2.6. 2.7, 2.8, 2.9, 3.0. 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5 8, 5.9, 6.0, 6.1, 6.2, 6.3, 6 4, 6.5, 6.6, 6.7, 6.8, 6.9, 7.0, 7.1, 7.2. 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11 6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3. 16.4, or 16.5 mg/kg of body weight, where a mg refers to the amount of IGF-1R ligand present in the conjugate.

In some embodiments, the conjugate of the present invention is administered at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight, where a mg refers to the amount of IGF-1R ligand present in the conjugate.

In some embodiments, the conjugate of the present invention is dosed at the maximum tolerated dose (MTD). “MTD,” as used herein, refers to the highest dose of an agent that an individual patient can tolerate. In other words, side effects in a given patient can determine the MTD. Side effects may limit the ability to administer higher doses of a treatment than the maximum tolerated dose. Therefore, the MTD for a given patient may be lower than those indicated in the prescribing information for the treatment or those commonly used in clinical practice. The MTD may have limited or no clinical efficacy in a patient.

In some embodiments, the conjugate of the present invention is administered daily, every other day, every three days, every four days, every five days, every six days, once per week, once every two weeks, once every three weeks, once every four weeks, once per month, every two months, or every three months.

The conjugate of the present invention can be administered by any convenient route, for example, by infusion or bolus injection, by absorption through epithelial or mucocutaneous linings (e.g., oral mucosa, rectal, and intestinal mucosa, etc.). Administration can be systemic or local. Various delivery systems are known, e.g., microparticles, microcapsules, capsules, etc., and may be useful for administering a composition of the invention. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The mode of administration is left to the discretion of the practitioner, and will depend, in-part, upon the site of the medical condition.

In some embodiments, the compositions of the invention are administered parenterally. In some embodiments, the compositions of the invention are administered intravenously. In another embodiment, the compositions of the invention are administered by continuous infusion. In a particular embodiment, the compositions of the invention are administered by an infusion that lasts for 15 minutes, 20 minutes, 30 minutes, 45 minutes, 1 hour, or 2 hours.

In some embodiments, it can be desirable to administer one or more compositions of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as sialastic membranes, or fibers. In certain embodiments, one or more compositions of the invention can be injected intraperitoneally.

In yet another embodiment, the compositions of the invention can be delivered in a controlled release system.

In some embodiments, a pump can be used to deliver the compositions of the invention (see, e.g, Sefton, CRC Crit. Ref. Biomed. Eng. 1987, 14, 201; Buchwald et al., Surgery 1980, 88:507; Saudek et al., N. Engl. J. Med. 1989, 321:574). In some embodiments, the pump may be, but is not limited to, an insulin-like pump.

In some embodiments, the conjugate of the present invention is administered intravenously, subcutaneously, via intravitreal injection, via intra-orbital injection, or via eye dropper.

In some embodiments, the method of the present invention does not cause unacceptable hyperglycemia in the subject. Hyperglycemia is another term for high blood glucose and can occur when there is insufficient insulin in the body or when the body cannot utilize insulin properly. Unacceptable hyperglycemia refers to an adverse effect of grade 3 or higher, as determined by a treating physician, and/or one that cannot be controlled with diabetic medications and leads to discontinuation of treatment with the conjugate of the present invention.

### Patient Populations

TED is a progressive condition with two distinct phases. The first phase is an acute or active inflammatory phase characterized by soft tissue expansion and lasting anywhere from 6 months to 3 years. The second phase is a chronic or inactive phase characterized by fibrosis and reduced inflammation. In some embodiments, the conjugate of the present invention is administered to a subject diagnosed with active TED, acute TED, inactive TED, or chronic TED.

In some embodiments, the conjugate of the present invention is administered to a subject diagnosed with moderate-to-severe TED or sight-threatening (very severe) TED. Moderate-to-severe TED includes patients without sight-threatening disease but whose eye disease has sufficient impact on daily life as to justify the risks of immunosuppression (if active disease) or surgical intervention (if inactive disease). These patients usually have two or more of the following: lid retraction ≥2 mm, moderate or severe soft tissue involvement, proptosis ≥3 mm above normal for race and gender, and inconstant or constant diplopia. Sight-threatening TED include patients with dysthyroid optic neuropathy and/or corneal breakdown. See Bartalena et al. Eur J Endocrinol. 185(4): G43-G67 (2021).

In some embodiments, the subject with TED has Grave's hyperthyroidism and/or has a CAS of at least 3 or at least 4.

In some embodiments, the subject having an inflammatory condition of the eye treated in accordance with the methods described herein has not previously received treatment for the inflammatory condition of the eye. In some embodiments, the subject having an inflammatory condition of the eye treated in accordance with the methods described herein has previously received treatment for the inflammatory condition of the eye.

In some embodiments, the previous treatment comprised administration of an IGF-1R inhibitor to the subject.

In some embodiments, the IGF-IR inhibitor was an antibody. In a specific embodiment, the antibody is teprotumumab, ganitumab, figitumumab, MEDI-573, cixutumumab, dalotuzumab, robatumumab, AVE1642, BIIB022, xentuzumab, istiratumab, lonigutamab, VB421, VRDN-001, VRDN-002, or VRDN-003.

In some embodiments, the previous treatment comprised administration of an antibody or antibody fragment targeting the neonatal Fc receptor (FcRn). In a specific embodiment, the antibody is batoclimab, IMVT-1402, or efgartigimod.

In some embodiments, the previous treatment comprised administration of an antibody targeting IL-6. In a specific embodiment, the antibody is TOUR006.

In some embodiments, the IGF-1R inhibitor was a small molecule. In a specific embodiment, the small molecule is linsitinib, picropodophyllin, AXL1717, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, or A-928605.

In some embodiments, the IGF-1R inhibitor was a radioimmunoconjugate. In a specific embodiment, the radioimmunoconjugate was FPI-1434.

In some embodiments, the IGF-1R inhibitor was an antibody-drug conjugate. In a specific embodiment, the antibody-drug conjugate was W0101.

In some embodiments, the subject has relapsed from previous treatment for the inflammatory condition of the eye.

In some embodiments, the subject was refractory to previous treatment for inflammatory condition of the eye. As used herein, a subject is “refractory” to prior treatment if the subject has failed to achieve a response to a therapy such that the therapy is determined to not be therapeutically effective, such as: failure to reach clinical endpoint, including any of response, extended duration of response, extended disease-free survival, relapse-free survival, and progression-free survival.

The subject matter described herein includes, but is not limited to, the following embodiments:
1. A method for treating an inflammatory condition of the eye in a subject, said method comprising administering to the subject an effective amount of a conjugate, wherein said conjugate comprises (i) an insulin-like growth factor 1 receptor (IGF-1R) ligand, or portion or variant thereof, and (ii) a disease-modifying agent.
2. The method of embodiment 1, wherein said inflammatory condition of the eye is thyroid eye disease (TED), uveitis, scleritis, keratitis, conjunctivitis, or an orbital inflammatory disease.
3. The method of embodiment 2, wherein said orbital inflammatory disease is idiopathic orbital inflammation, orbital inflammatory pseudotumor, orbital myositis, inflammatory orbital cellulitis, optic perineuritis, periscleritis, diffuse orbital inflammation, orbital apicitis, or sclerosing orbital inflammation.
4. Embodiment 2, wherein said inflammatory condition of the eye is TED.
5. The method of embodiment 4, wherein said TED is active TED, acute TED, inactive TED, chronic TED, moderate-to-severe TED, or sight-threatening (very severe) TED.
6. The method of embodiment 4 or 5, wherein said treatment results in a reduction in proptosis in said subject.
7. The method of embodiment 6, wherein proptosis is reduced by at least 2 mm, at least 3 mm, or at least 4 mm.
8. The method of embodiment 6 or 7, wherein said reduction in proptosis is assessed by exophthalmometer or orbital imaging.
9. The method of embodiment 8, wherein said orbital imaging is computerized tomography (CT) scan or magnetic resonance imaging.
10. The method of any one of embodiments 6-9, wherein the reduction in proptosis is associated with a reduction in extraocular muscle volume and/or orbital fat volume.
11. The method of any one of embodiments 4-10, wherein said treatment results in a reduction in the clinical activity score (CAS) in said subject.
12. The method of embodiment 11, wherein the CAS is reduced by at least 2 points or at least 3 points.
13. The method of embodiment 11, wherein the CAS is reduced to 1 or reduced to 0.
14. The method of any one of embodiments 4-13, wherein said treatment results in a reduction in the severity of diplopia in said subject.
15. The method of embodiment 14, wherein said reduction in the severity of diplopia is measured by the Gorman subjective diplopia score.
16. The method of embodiment 14 or 15, wherein said diplopia is constant diplopia, intermittent diplopia, or inconstant diplopia.
17. The method of any one of embodiments 14-16, wherein said treatment results in complete resolution of diplopia.
18. The method of any one of embodiments 15-17, wherein the severity of diplopia in the subject is reduced by at least one grade.
19. The method of any one of embodiments 4-18, wherein said treatment results in an improvement in the quality of life of said subject.
20. The method of embodiment 19, wherein said improvement in the quality of life is measured by the Graves' Ophthalmopathy Quality of Life (GO-QoL) assessment scale, or an improvement on the Visual Functioning subscale or the Appearance subscale of the GO-QoL.
21. The method of embodiment 20, wherein quality of life is improved by at least 8 points on the GO-QOL scale or the Visual Functioning subscale of the GO-QOL or the Appearance subscale of the GO-QoL.
22. The method of any one of embodiments 4-21, wherein said treatment results in an improvement in optic neuropathy.
23. The method of any one of embodiments 4-22, wherein said treatment results in a reduction in retro-orbital edema.
24. The method of any one of embodiments 4-23, wherein said treatment results in an improvement in monocular ductions.
25. The method of embodiment 24, wherein said improvement in monocular ductions is measured by the light reflex test.
26. The method of embodiment 25, wherein said improvement is of at least 10 degrees.
27. The method of any one of embodiments 1-26, wherein said treatment results in an alteration or impairment in the function of IGF-1R-expressing cells in said subject.
28. The method of any one of embodiments 1-26, wherein said treatment results in a reduction in the number of IGF-1R-expressing cells of said subject.
29. The method of embodiment 28, wherein said reduction is caused by killing of said IGF-1R-expressing cells.
30. The method of any one of embodiments 27-29, wherein said IGF-1R-expressing cells are orbital fibroblasts (OFs).
31. The method of any one of embodiments 4-30, wherein said treatment results in reduction or inhibition of hyaluronan synthesis in the retro-ocular space.
32. The method of any one of embodiments 4-31, wherein said treatment results in reduction or inhibition of adipogenesis in the retro-ocular space of said subject.
33. The method of any one of embodiments 4-32, wherein said treatment results in reduction in the level of interleukin (IL)-6, IL-16, and/or RANTES in the serum of said subject.
34. The method of any one of embodiments 27-29, wherein said IGF-1R-expressing cells are fibrocytes, B lymphocytes, and/or T lymphocytes.
35. The method of any one of embodiments 1-34, wherein the IGF-1R is overexpressed by retro-ocular space cells in said subject relative to retro-ocular space cells from a healthy subject or a subject that has not been diagnosed with said inflammatory condition of the eye.
36. The method of any one of embodiments 1-34, wherein the frequency of cells expressing IGF-1R in said subject is increased relative to cells from a healthy subject or a subject that has not been diagnosed with said inflammatory condition of the eye.
37. The method of embodiment 36, wherein said frequency of IGF-1R-expressing cells is measured by flow cytometry or immunohistochemistry.
38. The method of any one of embodiments 1-37, wherein said treatment results in disease modification in said subject.
39. The method of any one of embodiments 1-38, wherein said IGF-1R ligand comprises wildtype insulin-like growth factor 1 (IGF-1) (SEQ ID NO:3), wildtype insulin (SEQ ID NO:10 and SEQ ID NO: 11), or wildtype insulin-like growth factor 2 (IGF-2) (SEQ ID NO:12).
40. The method of any one of embodiments 1-38, wherein said IGF-1R ligand comprises a variant of wildtype IGF-1 (SEQ ID NO:3), a variant of wildtype insulin (SEQ ID NO:10 and SEQ ID NO: 11), or a variant of wildtype IGF-2 (SEQ ID NO:12).
41. The method of embodiment 40, wherein said variant of wildtype IGF-1 is at least 90% identical to IGF-1 (SEQ ID NO:3), said variant of wildtype insulin is at least 90% identical to insulin (SEQ ID NO: 10 and SEQ ID NO:11), or said variant of wildtype IGF-2 is at least 90% identical to IGF-2 (SEQ ID NO: 12).
42. The method of embodiments 40 or 41, wherein: (i) said variant of IGF-1 has reduced binding affinity for insulin-like growth factor binding proteins (IGFBPs) as compared to wildtype IGF-1 (SEQ ID NO:3) or said variant of IGF-2 has reduced binding affinity for IGFBPs as compared to wildtype IGF-2 (SEQ ID NO:12), and/or (ii) said variant of IGF-1 has increased affinity for the IGF-1R than wildtype IGF-1 (SEQ ID NO:3) or said variant of IGF-2 has increased affinity for the IGF-1R than wildtype IGF-2 (SEQ ID NO:12).
43. The method of any one of embodiments 1-38 or embodiments 40-42, wherein said IGF-1R ligand comprises 765IGF (SEQ ID NO:2), IGF-132 (SEQ ID NO:4), long-R3-IGF-1 (SEQ ID NO: 5), R3-IGF-1 (SEQ ID NO:6), des (1-3)-IGF-1 (SEQ ID NO:7), long-IGF-1 (SEQ ID NO:8), or long-G3-IGF-1 (SEQ ID NO:9).
44. The method of any one of embodiments 1-43, wherein said IGF-1R ligand, or portion or variant thereof, comprises a leader sequence.
45. The method of embodiment 44, wherein said leader sequence comprises SEQ ID NO:1.
46. The method of any one of embodiments 43-45, wherein said IGF-1R ligand comprises 765IGF (SEQ ID NO:2).
47. The method of any one of embodiments 1-46, wherein said disease-modifying agent comprises a cytotoxic agent.
48. The method of embodiment 47, wherein said cytotoxic agent comprises a chemotherapeutic agent.
49. The method of embodiment 48, wherein said chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine.

50. The method of embodiment 49, wherein said chemotherapeutic agent is methotrexate.
51. The method of embodiment 47, wherein said cytotoxic agent comprises a toxin.
52. The method of embodiment 51, wherein said toxin comprises Clostridium perfringens enterotoxin, diphtheria toxin, ricin chain A, Pseudomonas exotoxin, A chain toxins, a ribosome inactivating protein, α-sarcin, aspergillin, or a ribonuclease.
53. The method of embodiment 52, wherein said toxin comprises Clostridium perfringens enterotoxin, or a portion or variant thereof.
54. The method of embodiment 53, wherein the conjugate comprises SEQ ID NO: 14 or SEQ ID NO: 15.
55. The method of embodiment 52, wherein said toxin comprises diphtheria toxin, or a portion or variant thereof.
56. The method of embodiment 55, wherein the conjugate comprises SEQ ID NO: 13 or SEQ ID NO: 16.
57. The method of any one of embodiments 1-46, wherein said disease-modifying agent is selected from: glucocorticoids (dexamethasone); corticosteroids (methylprednisolone, prednisone, triamcinolone acetonide); a thyroid-stimulating hormone receptor (TSHR) inhibitor; mycophenolate mofetil; simvastatin; metformin; phenformin; cyclosporin; rapamycin or other mammalian target of rapamycin (mTOR) inhibitors; or azathioprine.
58. The method of any one of embodiments 4-57, wherein said subject (i) has Grave's hyperthyroidism; and/or (ii) has a CAS of at least 3 or at least 4.
59. The method of any one of embodiments 1-58, wherein said subject (i) has not previously received treatment for said inflammatory condition of the eye; (ii) has previously received treatment for said inflammatory condition of the eye; (iii) has relapsed from previous treatment for said inflammatory condition of the eye; or (iv) was refractory to previous treatment for said inflammatory condition of the eye.
60. The method of embodiment 59, wherein said previous treatment comprised administration of an IGF-1R inhibitor to said subject.
61. The method of embodiment 60, wherein said IGF-1R inhibitor was an antibody.
62. The method of embodiment 61, wherein said antibody is teprotumumab, ganitumab, figitumumab, MEDI-573, cixutumumab, dalotuzumab, robatumumab, AVE1642, BIIB022, xentuzumab, istiratumab, lonigutamab, VB421, VRDN-001, VRDN-002, or VRDN-003.
63. The method of embodiment 60, wherein said IGF-1R inhibitor was a small molecule.
64. The method of embodiment 63, wherein said small molecule is linsitinib, picropodophyllin, AXL1717, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, or A-928605.
65. The method of embodiment 60, wherein said IGF-IR inhibitor was a radioimmunoconjugate.
66. The method of embodiment 65, wherein radioimmunoconjugate was FPI-1434.
67. The method of embodiment 60, wherein said IGF-1R inhibitor was an antibody-drug conjugate.
68. The method of embodiment 67, wherein antibody-drug conjugate was W0101.
69. The method of any one of embodiments 1-68, wherein said conjugate is administered in combination with one or more other therapies.
70. The method of embodiment 69, wherein said one or more other therapies comprises one or more of the following: glucocorticoids (dexamethasone); corticosteroids (methylprednisolone, prednisone, triamcinolone acetonide); rituximab or other anti-CD20 antibodies; tocilizumab, TOUR006, or other anti-IL-6 antibodies; selenium; infliximab, adalimumab, or other anti-tumor necrosis factor (TNF) a antibodies; a TSHR inhibitor; orbital radiotherapy; a vascular endothelial growth factor (VEGF) inhibitor; mycophenolate mofetil; iscalimab or other anti-CD40 antibodies; IMVT-1401, batoclimab, efgartigimod, or other fragment crystallizable neonatal receptor (FcRN) inhibitors; simvastatin; metformin; phenformin; cyclosporin; azathioprine; methotrexate; rapamycin or mTOR inhibitors; and/or intravenous immunoglobulin treatment.
71. The method of any one of embodiments 1-50 or embodiment 57, wherein said conjugate is administered at dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.
72. The method of any one of embodiments 1-71, wherein said conjugate is administered at dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2. 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15 7, 15.8, 15.9, 16.0, 16 1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.
73. The method of any one of embodiments 1-70, wherein said conjugate is administered at a dose that is the maximum tolerated dose.
74. The method of any one of embodiments 1-73, wherein said conjugate is administered daily, every other day, every three days, every four days, every five days, every six days, once per week, once every two weeks, once every three weeks, once every four weeks, once per month, every two months, or every three months.
75. The method of any one of embodiments 1-74, wherein said conjugate is administered intravenously, subcutaneously, via intravitreal injection, via intra-orbital injection, or via eye dropper.
76. The method of any one of embodiments 1-75, wherein said method does not cause unacceptable hyperglycemia in said subject.
77. A method for treating TED in a subject, said method comprising administering to the subject an effective amount of a conjugate, wherein said conjugate comprises (i) 765IGF (SEQ ID NO:2) and (ii) methotrexate.

The following examples are offered by way of illustration and not by way of limitation.

### EXAMPLES

### Example 1: Assessment of LX-101 Efficacy on TED Orbital Fibroblasts

LX-101 is an IGF-1R ligand (SEQ ID NO: 2) conjugate with methotrexate, a well-known inhibitor of dihydrofolate reductase, which ultimately inhibits nucleotide synthesis. Without wishing to be bound by theory, LX-101 may deliver methotrexate to cells overexpressing IGF-1R implicated in the pathology of TED and reduce their viability and proliferation. Primary orbital fibroblasts (OFs) are obtained from patients with TED undergoing orbital decompression surgery and cultured with LX-101 at a range of doses up to 2500 nM (expressed in terms of IGF-1 protein). For comparison, some OFs are treated with IGF-1 to activate canonical IGF-1R signaling. Viability is determined using an Alamar blue assay or any viability assay known in the art. Proliferation is determined using BrdU incorporation or any proliferation assay known in the art.

OFs may be treated with cytokines for activation, including platelet-derived growth factor BB (PDGF-BB), which stimulates OF proliferation, and transforming growth factor β (TGF-62 ), which promotes OF differentiation into myofibroblasts and scar-tissue formation. Additional activators of OFs include IL-1, IL-4, IL-6, IL-17, leukoregulin, IGF-1, TNF-α, IFN-γ, CD40L, PPARy agonists, and autoantibodies from Graves' Disease patients (e.g., autoantibodies to TSHR). The effect of LX-101 on activated OFs is studied. Without wishing to be bound by theory, LX-101 may have a more significant effect on activated OFs, which are relevant to TED pathology.

For one cell viability assay, OFs are plated in black 96-well plates with culture medium along with the appropriate treatment. Puromycin is used as a positive control for cytotoxicity. Cells are incubated with treatments for a period of time, then Alamar blue reagent is added to each well. After incubation, fluorescence of the oxidized Alamar reagent is measured (excitation, 470 nm; emission, 480 nm) and normalized to vehicle-treated cells.

For one cell proliferation assay, OF strains are seeded in a 96-well plate. Cells are treated in triplicate with specific treatments for a period of time (e.g., 2-14 days) before addition of bromodeoxyuridine (BrdU) for 18 to 24 hours, and proliferation is measured using a BrdU cell proliferation assay kit. Samples are fixed and stained with an anti-BrdU antibody, incubated with a corresponding horseradish peroxidase (HRP) conjugated secondary antibody, and assessed using a microplate reader.

Without wishing to be bound by theory, LX-101 may reduce the production of hyaluronic acid (HA) produced by OFs, which contributes to the pathology of TED. To detect and analyze HA production, supernatants are collected from OFs after treatment with LX-101 and the amount of HA in each sample is measured by ELISA.

IL-6, a proinflammatory cytokine produced by lymphocytes, monocytes, as well as OFs, is present in high concentrations in TED patients and plays an important role in the pathogenesis of the disease. Without wishing to be bound by theory, LX-101 may reduce the production of IL-6. To detect and analyze IL-6 production, supernatants are collected from OFs after treatment with LX-101 and the amount of IL-6 in each sample is measured by ELISA.

Without wishing to be bound by theory, LX-101 may inhibit the inflammatory phenotype of OFs. ELISA and Western blot can be used to detect and analyze molecular mediators of inflammation (e.g., COX-2).

The effect of LX-101 in OFs from non-TED patients and/or fibroblasts from other tissues (e.g., eyelid or dermal fibroblasts) can also be studied and compared.

As TED is a complex disease involving a variety of cells, the effect of LX-101 on OFs grown in co-culture with immune cells is studied. Lymphocytes play a significant role in the early stages of TED and may initiate inflammatory processes and cytokine production. OFs are grown in co-culture with B cells and/or T cells from TED patients to study the effect of LX-101 on OF viability, proliferation, and phenotype.

### Example 2: Investigating the Mechanism of Action of LX-101 in TED OFs

Primary OFs from patients with TED are cultured and treated with varying doses of LX-101 (e.g., 2500-1.64 nM, expressed in terms of IGF-1 protein) for a period of time (e.g., 2-14 days). The specificity of LX-101 targeting IGF-1R can be studied by treating OFs with siRNA specific to IGF-IR to deplete IGF-IR or with a non-specific siRNA control. Without wishing to be bound by theory, the effect of LX-101 on OFs may be abrogated by depletion of IGF-IR expression.

The binding affinity of LX-101 to IGF-IR on TED OFs and competition with endogenous ligands such as IGF-1 is characterized. The effect of LX-101 on IGF-1R signaling pathways is determined by detecting phosphorylation of intracellular signaling proteins, such as PI3K, AKT, MAPK, and JNK, such as by Western blot. The specificity of LX-101 to target IGF-1R signaling can be determined by depleting IGF-1R as described above. Additionally, it is determined if LX-101 induces apoptosis in TED OFs.

### Example 3: Investigating the Effect of LX-101 Against Adipocytes and Myofibroblasts Differentiated From TED OFs

Primary OFs from patients with TED are treated with varying doses of LX-101 (e.g., 2500-1.64 nM, expressed in terms of IGF-1 protein) for a period of time (e.g., 2-14 days) and further cultured to differentiate into myofibroblasts or adipocytes. The OFs are cultured to differentiate into myofibroblasts by the addition of TGF-B, with and without the addition of IGF-1. The effects of myofibroblast formation and activation in the presence of LX-101 are measured by Western blot and RT-qPCR (e.g., for targets such as smooth muscle actin, collagen, and fibronectin), immunofluorescence (e.g., for the detection of actin filament formation, HA accumulation), and ELISA (HA production).

Additionally, OFs are cultured to differentiate into adipocytes using a standard adipogenic medium. Adipocyte formation is measured by Western blot, RT-qPCR, and immunofluorescence for markers of adipogenesis (e.g., FABP4, PPARγ, lipid accumulation). The effect of LX-101 on OFs from patients who did not respond to teprotumumab are also tested. Thus, the role of LX-101 in targeting myofibroblast and adipocyte differentiation is studied. Without wishing to be bound by theory, LX-101 may inhibit the ability of OFs to differentiate into myofibroblasts and adipocytes and may also directly inhibit the viability of differentiated myofibroblasts and adipocytes.

### Example 4: Investigating the Effect of LX-101 on the Viability of Circulating PBMCs From TED Patients

Peripheral blood mononuclear cells (PBMCs) from patents with TED are obtained and treated with varying doses of LX-101 (e.g., 2500-1.64 nM, expressed in terms of IGF-1 protein) for a period of time (e.g., 2-14 days) and evaluated by flow cytometry. The cells are identified by CD markers (e.g., CD34 for fibrocytes, CD20 for B cells, CD3 for T cells, and CD14 for monocytes). Cell viability is measured by 7-AAD staining or any method known in the art and proliferation is measured by BrdU labeling or any method known in the art.

In some PBMC cultures, T cells are activated using anti-CD28/CD3 magnetic beads and recombinant IL-2. Expanded T cells are treated with IGF-1 +/−LX-101 and viability and proliferation are measured. The ability of LX-101 to target additional cell types involved in the pathophysiology of TED is determined. Without wishing to be bound by theory, LX-101 may inhibit the viability and/or proliferation of fibrocytes, T cells, and B cells from TED patients. Additionally, it will be determined if LX-101 targets IGF-1 signaling in T cells from TED patients.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical, and scientific terms used herein have the same meaning as commonly understood by one of ordinary skill in the art to which this subject matter belongs.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for treating an inflammatory condition of the eye in a subject in need thereof, said method comprising administering to the subject a conjugate comprising an insulin-like growth factor 1 receptor (IGF-1R) ligand, or portion or variant thereof, and a disease-modifying agent.

2. The method of claim 1, wherein said inflammatory condition of the eye is selected from the group consisting of thyroid eye disease (TED), uveitis, scleritis, keratitis, conjunctivitis, and an orbital inflammatory disease.

3. The method of claim 2, wherein said orbital inflammatory disease is selected from the group consisting of idiopathic orbital inflammation, orbital inflammatory pseudotumor, orbital myositis, inflammatory orbital cellulitis, optic perineuritis, periscleritis, diffuse orbital inflammation, orbital apicitis, and sclerosing orbital inflammation.

4. The method of claim 2, wherein said inflammatory condition of the eye is TED.

5. The method of claim 4, wherein said TED is selected from the group consisting of active TED, acute TED, inactive TED, chronic TED, moderate-to-severe TED, and sight-threatening TED.

6. The method of claim 4 or 5, wherein said method results in a reduction in proptosis in said subject.

7. The method of claim 6, wherein proptosis is reduced by at least 2 mm, at least 3 mm, or at least 4 mm.

8. The method of claim 6 or 7, wherein said reduction in proptosis is assessed by exophthalmometer or orbital imaging.

9. The method of claim 8, wherein said orbital imaging is computerized tomography (CT) scan or magnetic resonance imaging.

10. The method of any one of claims 6-9, wherein the reduction in proptosis is associated with a reduction in extraocular muscle volume.

11. The method of any one of claims 6-9, wherein the reduction in proptosis is associated with a reduction in orbital fat volume.

12. The method of any one of claims 4-11, wherein said method results in a reduction in the clinical activity score (CAS) in said subject.

13. The method of claim 12, wherein the CAS is reduced by at least 2 points or at least 3 points.

14. The method of claim 12, wherein the CAS is reduced to 1 or reduced to 0.

15. The method of any one of claims 4-14, wherein said method results in a reduction in the severity of diplopia in said subject.

16. The method of claim 15, wherein said reduction in the severity of diplopia is measured by a Gorman subjective diplopia score.

17. The method of claim 15 or 16, wherein said diplopia is selected from the group consisting of constant diplopia, intermittent diplopia, and inconstant diplopia.

18. The method of any one of claims 15-17, wherein said method results in complete resolution of diplopia.

19. The method of any one of claims 16-18, wherein the severity of diplopia in the subject is reduced by at least one grade.

20. The method of any one of claims 4-19, wherein said method results in an improvement in the quality of life of said subject.

21. The method of claim 20, wherein said improvement in the quality of life is measured by a Graves' Ophthalmopathy Quality of Life (GO-QoL) assessment scale.

22. The method of claim 20 or 21, wherein said quality of life is measured by a Visual Functioning subscale or an Appearance subscale of the GO-QoL.

23. The method of claim 21 or 22, wherein said quality of life is improved by at least 8 points.

24. The method of any one of claims 4-23, wherein said method results in an improvement in optic neuropathy.

25. The method of any one of claims 4-24, wherein said method results in a reduction in retro-orbital edema.

26. The method of any one of claims 4-25, wherein said method results in an improvement in monocular ductions.

27. The method of claim 26, wherein said improvement in monocular ductions is measured by a light reflex test.

28. The method of claim 27, wherein said improvement is of at least 10 degrees.

29. The method of any one of claims 1-28, wherein said subject has a reduction in a number of IGF-1R expressing cells.

30. The method of claim 29, wherein the reduction in the number of IGF-IR-expressing cells is measured by flow cytometry or immunohistochemistry.

31. The method of claim 29 or 30, wherein said IGF-1R-expressing cells are orbital fibroblasts (OFs).

32. The method of any one of claims 4-31, wherein said method results in reduction or inhibition of hyaluronan synthesis in a retro-ocular space.

33. The method of any one of claims 4-32, wherein said method results in reduction or inhibition of adipogenesis or a reduction in adipocytes in the retro-ocular space of said subject.

34. The method of any one of claims 4-33, wherein said method results in reduction in the level of interleukin (IL)-6, IL-16, and/or RANTES in the serum of said subject.

35. The method of claim 29 or 30, wherein said IGF-1R-expressing cells are selected from the group consisting of fibrocytes, B lymphocytes, and T lymphocytes.

36. The method of any one of claims 1-35, wherein said IGF-1R ligand comprises wildtype insulin-like growth factor 1 (IGF-1), wildtype insulin, or wildtype insulin-like growth factor 2 (IGF-2).

37. The method of claim 36, wherein said wildtype IGF-1 comprises SEQ ID NO:3, wherein said wildtype insulin comprises SEQ ID NO: 10 or 11, and wherein said wildtype IGF-2 comprises SEQ ID NO:12.

38. The method of any one of claims 1-37, wherein said IGF-1R ligand comprises a variant of wildtype IGF-1, a variant of wildtype insulin, or a variant of wildtype IGF-2.

39. The method of claim 38, wherein said variant of wildtype IGF-1 is at least 90% identical to SEQ ID NO:3, said variant of wildtype insulin is at least 90% identical to SEQ ID NO: 10 or 11, and said variant of wildtype IGF-2 is at least 90% identical to SEQ ID NO:12.

40. The method of claim 38 or 39, wherein: (i) said variant of wildtype IGF-1 has reduced binding affinity for insulin-like growth factor binding proteins (IGFBPs) as compared to wildtype IGF-1, or said variant of wildtype IGF-2 has reduced binding affinity for IGFBPs as compared to wildtype IGF-2, and/or (ii) said variant of wildtype IGF-1 has increased affinity for the IGF-1R as compared to wildtype IGF-1 or said variant of wildtype IGF-2 has increased affinity for the IGF-1R as compared to wildtype IGF-2.

41. The method of any one of claims 1-40, wherein said IGF-1R ligand, or portion or variant thereof, comprises a leader sequence.

42. The method of claim 41, wherein said leader sequence comprises SEQ ID NO:1.

43. The method of any one of claims 1-35 and claims 38-42, wherein said IGF-1R ligand comprises 765IGF (SEQ ID NO:2), IGF-132 (SEQ ID NO:4), long-R3-IGF-1 (SEQ ID NO:5), R3-IGF-1 (SEQ ID NO:6), des(1-3)-IGF-1 (SEQ ID NO:7), long-IGF-1 (SEQ ID NO:8), or long-G3-IGF-1 (SEQ ID NO:9).

44. The method of claim 43, wherein said IGF-1R ligand comprises 765IGF (SEQ ID NO:2).

45. The method of any one of claims 1-44, wherein the IGF-1R ligand, or portion or variant thereof, is covalently bound to said disease-modifying agent, wherein said disease-modifying agent comprises a cytotoxic agent.

46. The method of claim 45, wherein said cytotoxic agent comprises a chemotherapeutic agent.

47. The method of claim 46, wherein said chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine.

48. The method of claim 47, wherein said chemotherapeutic agent is methotrexate.

49. The method of claim 45, wherein said cytotoxic agent comprises a toxin.

50. The method of claim 49, wherein said toxin comprises Clostridium perfringens enterotoxin, diphtheria toxin, ricin chain A, Pseudomonas exotoxin, A chain toxins, a ribosome inactivating protein, α-sarcin, aspergillin, or a ribonuclease.

51. The method of claim 50, wherein said toxin comprises Clostridium perfringens enterotoxin, or a portion or variant thereof.

52. The method of claim 51, wherein the conjugate comprises SEQ ID NO:14 or SEQ ID NO: 15.

53. The method of claim 50, wherein said toxin comprises diphtheria toxin, or a portion or variant thereof.

54. The method of claim 53, wherein the toxin comprises SEQ ID NO: 13 or SEQ ID NO:16.

55. The method of any one of claims 1-44, wherein said disease-modifying agent is selected from the group consisting of glucocorticoids, corticosteroids, a thyroid-stimulating hormone receptor (TSHR) inhibitor, mycophenolate mofetil, simvastatin, metformin, phenformin, cyclosporin, rapamycin, mammalian target of rapamycin (mTOR) inhibitors, and azathioprine.

56. The method of any one of claims 1-55, wherein said conjugate is administered at dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.

57. The method of any one of claims 1-56, wherein said conjugate is administered at dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3. 5.4, 5.5, 5.6, 5.7, 5.8, 5.9. 6.0, 6.1, 6 2, 6.3, 6.4, 6.5. 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11. 1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13 2, 13.3, 13 4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.

58. The method of any one of claims 1-57, wherein said conjugate is administered at dose that is the maximum tolerated dose.

59. The method of any one of claims 1-58, wherein said conjugate is administered daily, every other day, every three days, every four days, every five days, every six days, once per week, once every two weeks, once every three weeks, once every four weeks, once per month, every two months, or every three months.

60. The method of any one of claims 1-59, wherein said conjugate is administered intravenously, subcutaneously, via intravitreal injection, via intra-orbital injection, or via eye dropper.

61. The method of any one of claims 1-35 and 38-60, wherein the IGF-IR ligand is SEQ ID NO: 2, the cytotoxic agent is methotrexate, wherein there are 6 to 10 methotrexate molecules for every IGF-1R ligand of SEQ ID NO:2, and the inflammatory condition of the eye is TED.
